(19) United States
(12) Patent Application Publication
(10) Pub. No.: US 2005/0096688 A1
(43) Pub. Date: May 5, 2005

(54) Gripper for catheter shaft

(76) Inventors: Robert Slazas, Miami, FL (US); Gregg Kenny, Wakefield, MA (US); William Lee, Wheaton, IL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/696,976

(22) Filed: Oct. 30, 2003

(51) Int. Cl. A61M 29/00

(52) U.S. Cl. 606/191; 606/194

(57) ABSTRACT

A gripper for a catheter shaft is a flexible tube surrounding a portion of the catheter shaft. A physician can move the gripper to a selected position on the catheter shaft, and then squeeze the outer surfaces of the gripper, which resiliently collapses around the catheter shaft. When squeezed, an inner surface of the gripper contacts the catheter shaft and can transmit forces applied by the physician to the outside of the gripper, including twisting of the catheter shaft or longitudinal pushing or pulling. The physician may then release the squeezing pressure, and has the option to move the gripper along the catheter shaft to a different position, and may choose to use the gripper at the new position by squeezing it again.

### BACKGROUND AND SUMMARY OF THE INVENTION

1. Technical Background

The present invention relates generally to medical devices, and more particularly to a gripper for use with a catheter shaft.

2. Discussion

Catheters are used in a wide variety of therapeutic medical applications. They are often relatively long, on the order of two to five feet in length. They are often relatively thin and flexible, having an outer dimension on the order of several millimeters or less. When a physician wants to manipulate the catheter by grasping a portion of the catheter shaft and moving it, the physician may sometimes desire a better grip on the catheter shaft. This may be particularly true if that portion of the catheter shaft is wet, or has saline or body fluids on it. In addition, the catheter shaft may have any of a variety of coatings, including various lubricious coatings such as a hydrophilic coating.

By way of example, the present invention will be partly described in relation to angioplasty treatments, in which a balloon catheter is used. However, it should be understood that the present invention relates to any catheter having a gripper for use with the catheter shaft according to the present invention as recited in the following claims, and it is not limited to angioplasty.

Most catheters have a relatively long and flexible tubular shaft defining one or more passages or “lumens,” and have a hub fitting attached at one end of the shaft for connecting the lumen or lumens defined by the shaft to various other equipment. This end of the catheter where he hub is located is customarily referred as the “proximal” end, while the other end is called the “distal” end.

The proximal hub usually will be provided with one or more ports communicating with the lumen or lumens, and each port will often be fitted with a luer-lock fitting, for providing a sealing coupling with other equipment. Another possible fitting for a port defined by the hub is a flexible valve for resiliently resisting the escape or entry of fluids into the corresponding lumen.

The hub is often designed to have a shape that has a greater dimension in one transverse direction than another. In other words, an example of a hub design is a hub with “wings” as is illustrated in FIGS. 1 and 2. Another style of hub is illustrated in FIGS. 3 and 4. This greater size in one direction or another allows the physician to grasp and twist the hub to impart torsional movement to the catheter shaft, as well as longitudinal or positional movement.

The proximal end of the hub is often fitted with a tubular strain relief which surrounds a short portion of the catheter shaft. The strain relief provides a flexibility transition between the proximal hub which is relatively stiff and the catheter shaft which is relatively flexible. The strain relief resists the possibility of the catheter shaft kinking at the transition between the proximal hub and the catheter shaft.

Examples of catheter hubs are shown in the following patents, which are co-owned with the present invention: U.S. Pat. No. 6,289,568 entitled “Method for Making A Balloon Catheter Stent Deployment System” issued to Miller, et al. on Sep. 18, 2001, U.S. Pat. No. 6,293,959, entitled “Balloon Catheter and Stent Delivery System Having Enhanced Stent Retention and Method,” issued to Miller et al. on Sep. 25, 2001, and U.S. Pat. No. 6,322,534, entitled “Variable Stiffness Balloon Catheter,” issued to Shkolnik on Nov. 27, 2001.

Common treatment methods for using such a catheter include advancing a guidewire into the body of a patient, by directing the guidewire distal end percutaneously through an incision and along a body passage until it is located within or beyond a desired site. The guidewire may be advanced before, or simultaneously with the catheter.

A more primitive form of a gripper has been provided with a type of medical device other than catheters known as guidewires. These grippers were generally provided separately, or were not initially installed on the guidewire. Such guidewire grippers generally had a channel for placing a section of the guidewire into the gripper channel, as well as a chuck and screw arrangement for tightening the chuck of the gripper onto the guidewire. If a physician desires to move this kind of guidewire gripper, they must unscrew and release the chuck before moving the gripper to a different position along the guidewire. This unscrewing operation must generally be done with two hands; this kind of guidewire gripper effectively provides a single fixed holding position on the guidewire that is difficult or inefficient to move to another holding position.

It is desirable to provide a gripper for a catheter shaft that provides an effective improved gripping surface, which is also easy and intuitive to use for gripping the catheter shaft, and also easy to move along the catheter shaft in between desired gripping positions. In addition, it is desirable to provide such an improved gripper that is integral with the catheter system, may be concentric to the shaft, and does not need to be separately or independently installed. It is also desirable to provide a gripper that may or may not be used, at the physician's discretion, and is provided in an initial storage position such that it is not in the physician's way if the physician chooses not to use it.

These and various other objects, advantages and features of the present invention will become apparent from the following description and claims, when considered in conjunction with the appended drawings.

### BRIEF DESCRIPTION OF THE DRAWINGS

FIG. 1 is an external perspective view of a catheter system with a gripper;

FIG. 2 is a partial external perspective view of the components of FIG. 1, in a different configuration;

FIG. 3 is a partial external perspective view of a catheter shaft, hub, gripper and guidewire;

FIG. 4 is a partial external perspective view of the components of FIG. 3, in a different configuration;

FIG. 5 is a cross-section view of a catheter shaft, hub and a gripper;

FIG. 6 is a cross-section view of a gripper;

FIG. 7 is a top view of a hub and partial catheter shaft;

FIG. 8 is a partial cross-section view of a catheter system; and

FIGS. 9 and 10 are partial cross-section views of grippers.

### DETAILED DESCRIPTION OF THE PREFERRED EMBODIMENT

The following description of the preferred embodiments of the present invention is merely illustrative in nature, and as such it does not limit in any way the present invention, its applications, or uses. Numerous modifications may be made by those skilled in the art without departing from the true spirit and scope of the invention.

Referring to the drawings, a gripper and catheter system is shown, with one of the preferred embodiments of the present invention being shown generally at reference numeral 10.

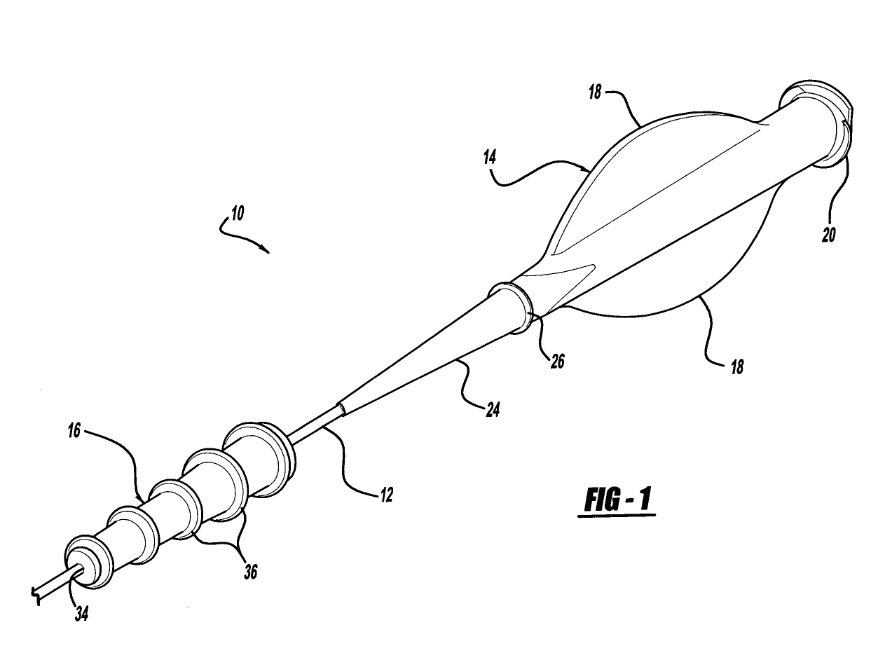

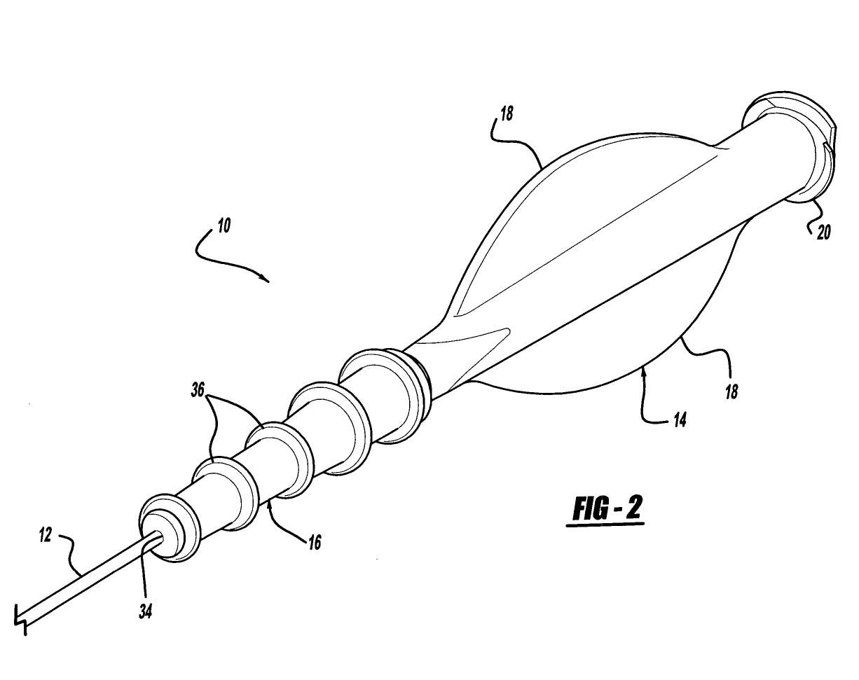

The catheter system 10 in FIGS. 1 and 2 has a catheter shaft 12, a hub 14 affixed to a proximal end of catheter shaft 12, and a tubular gripper 16 positioned around a portion of the catheter shaft 12. The hub may have some kind of outward extension for providing a physician with a good grip of the proximal end of the shaft, for example the pair of radially extending wings 18 shown in FIGS. 1 and 2. The proximal end of the hub may have a luer-lock fitting 20 and define a proximal port 22. A flexible strain relief 24 may be provided. The strain relief 24 is affixed to the hub at a transition between the hub 14 and the catheter shaft 12. Strain relief 24 may be provided with a circumferential bump 26.

The gripper 16 is tubular in shape, and has inner and outer surfaces 28 and 30. Gripper 16 defines a proximal and distal port 32 and 34, and may be provided with circumferential ridges 36, or any other features that may provide for a better grip.

The gripper may be provided with an internal indentation 38, for cooperating with the bump 26 on strain relief 24, so that the gripper 16 may be provided in an initial docked configuration as shown in FIG. 2. If the physician prefers not to use the gripper 16 at all, the gripper 16 may be left in this initial docked position, where it is out of the physician's way, and also may provide additional support to the strain relief 24. If the physician desires to use the gripper, it may be forcibly moved from this initial docked position, by overcoming the frictional force between the bump 26 and the indentation 38.

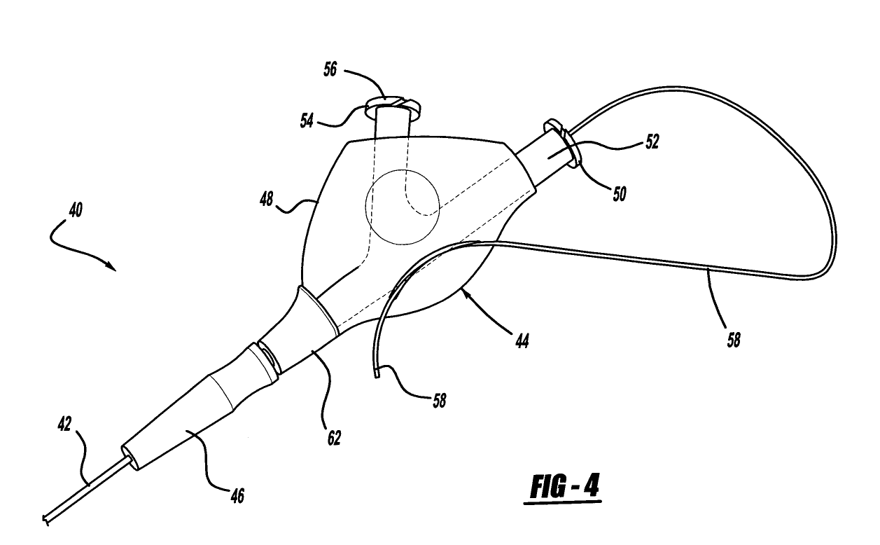

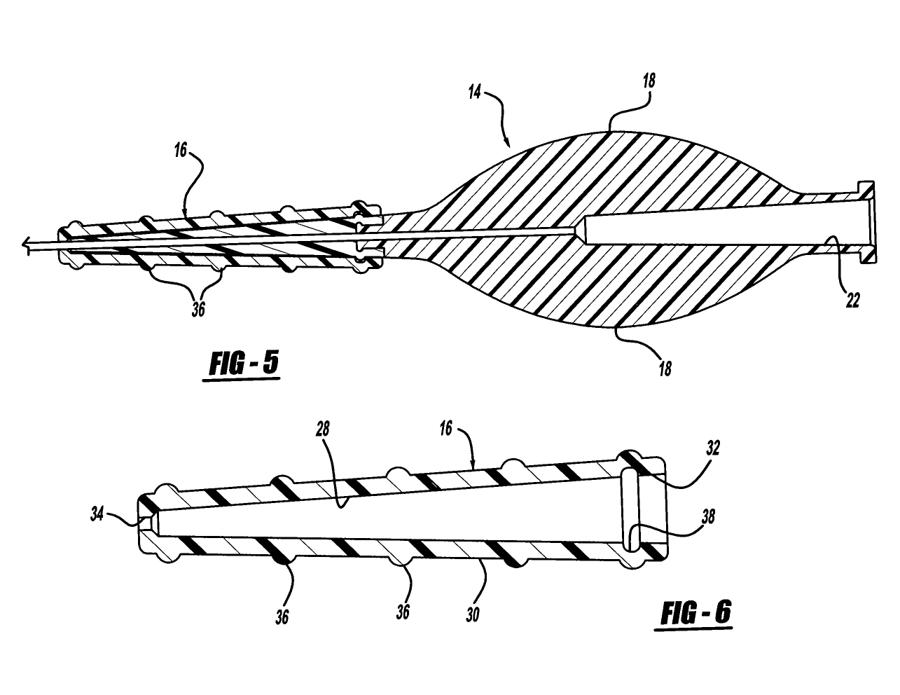

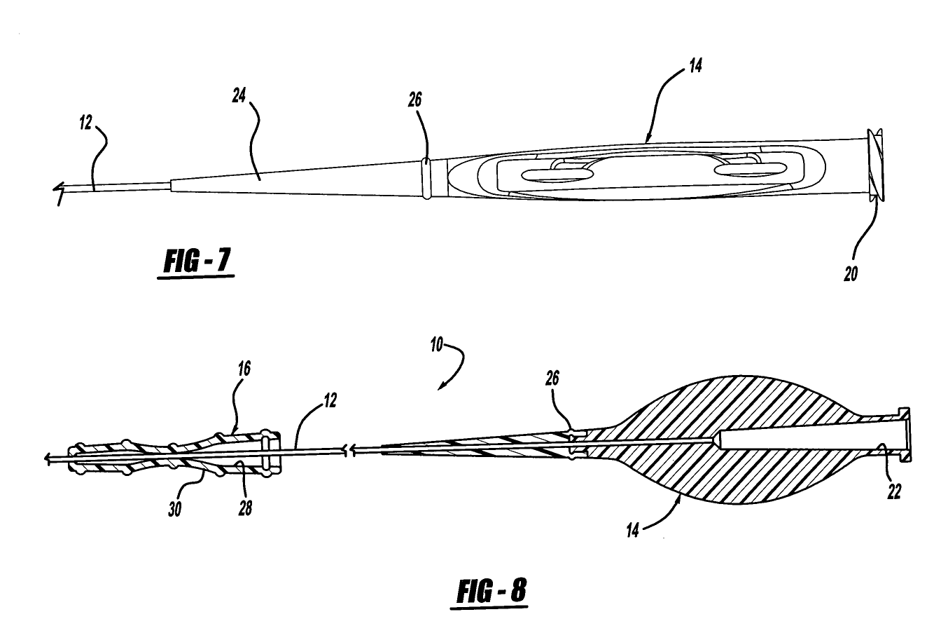

In operation, the gripper 16 may be moved from the initial docked position shown in FIG. 4 to a new position on the catheter shaft 12, where a better grip is desired by the physician. At this desired gripping position, the physician may squeeze the outer surface 30 of the gripper 16, which will tend to cause a portion of the gripper to resiliently compress such that a portion of the inner gripper surface 28 contacts the catheter shaft 12. In this position, the physician may apply enhanced movement forces onto the catheter shaft by manipulating the gripper 16.

In other words, the gripper of the present invention may be provided in an optional use configuration, such that the physician may use the gripper or ignore the gripper in any particular medical procedure.

One example of the present invention is a gripper 16 which may be used in connection with a balloon catheter, in which case the catheter shaft 12 defines an inflation lumen or passage, which is in communication with the hub proximal port 22, which in such event becomes a proximal inflation port.

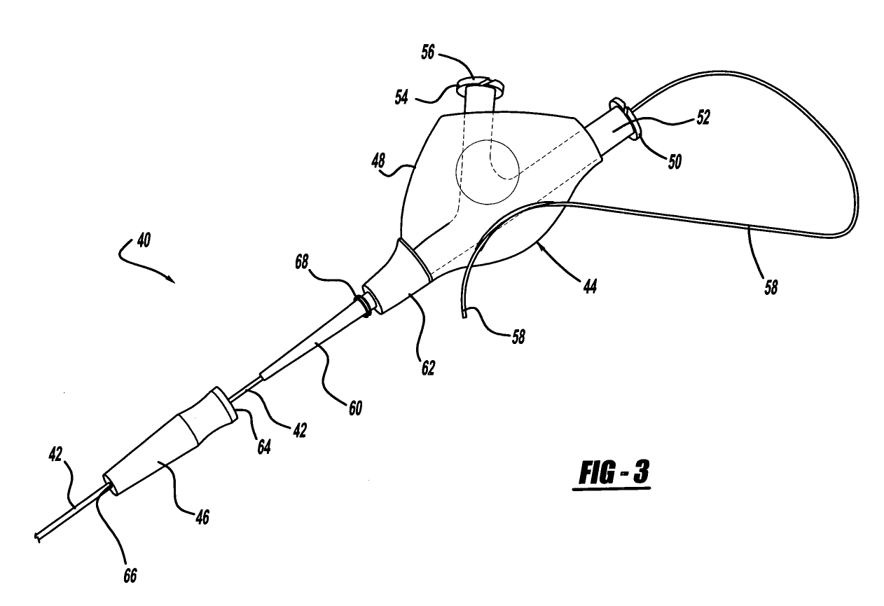

Another arrangement of the present invention is shown in FIGS. 3 and 4, in which the catheter shaft defines a first and second lumen or passage, (not shown), which respectively communicates with a first and second port defined by a hub. In FIGS. 3 and 4, catheter system 40 includes catheter shaft 42, hub 44 which is affixed to a proximal end of catheter shaft 42, and tubular gripper 46. The hub 44 again provides a radially extending portion 48 for more successfully manipulating the proximal end of the catheter shaft 42. A first luer-lock fitting 50 may be provided, or a haemostatic valve may be provided instead, communicating with a guidewire port 52. A second luer-lock fitting 54 defines an inflation port 56. A guidwire 58 is shown extending approximately from the proximal guidewire port. A flexible strain relief 60 having a strain relief mount 62 is affixed at the transition between the hub 44 and the catheter shaft 42. A circumferential bump 68 is provided.

Gripper 46 has a proximal and distal port 64 and 66. FIGS. 3 and 4 are intended to provide another example of a gripper according to the present invention, in this case with a proximal hub that has two ports. Of course the present invention relates to any tubular gripper used with a catheter system having features of the present invention, and is not limited to balloon catheters or particular kind of hub.

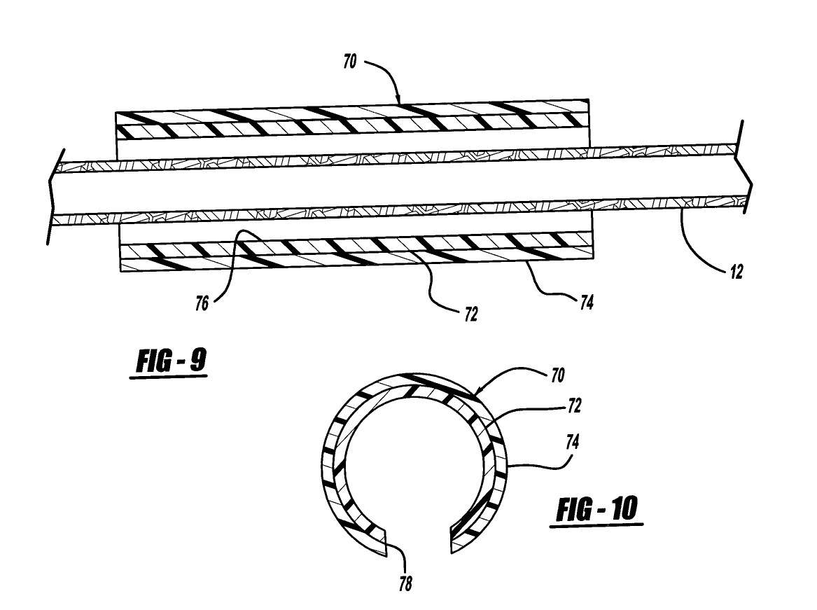

In the configuration shown in FIG. 8, the gripper 16 is depicted in a desired gripping position along the catheter shaft 12. A squeezing pressure is being applied to the gripper 16, and a portion of the gripper inner surface 28 is contacting the catheter shaft 12. Accordingly, movement forces applied by a physician to the outer surface 30 of the gripper 16 will be transferred more effectively to the catheter shaft 12. The physician is thus provided with a better grip and ability to move the catheter 10 to treat the patient.

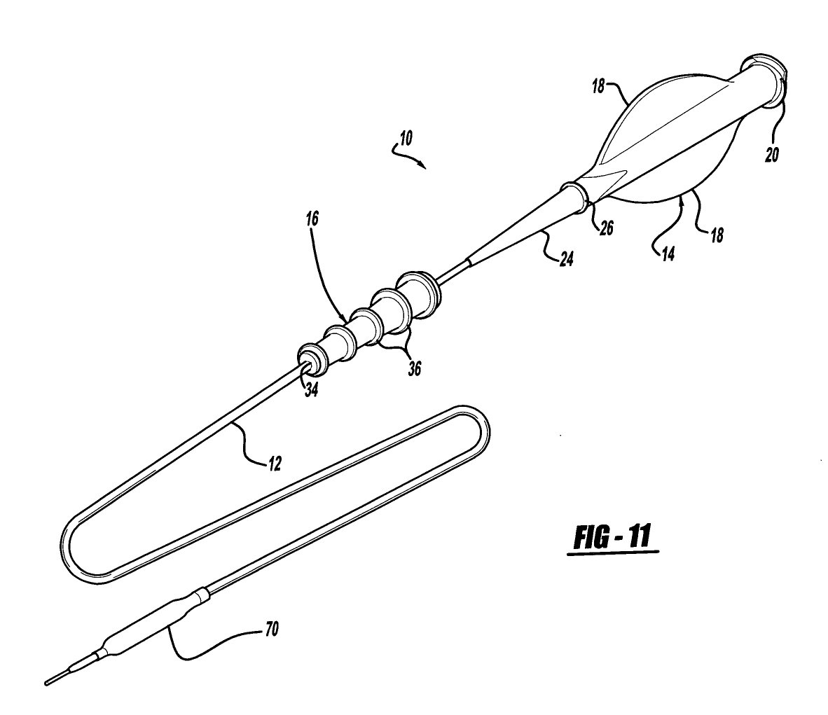

Another arrangement of the present invention is shown in FIGS. 9 and 10, in which the gripper 70 has multiple layers of material. In the embodiment depicted in FIGS. 9 and 10, an inner and outer layer 72 and 74 are shown. More layers of material are of course possible. One possible selection of materials for a multi-layer gripper is to provide an inner layer having a very high co-efficient of friction, and an outer layer for providing greater strain relief.

FIG. 10 also illustrates a desirable gap 76 defined between a default inner dimension of the gripper 70 and the catheter shaft 12. This gap 76 may be small, but its presence facilitates easy movement of the gripper along the shaft 12.

FIG. 10 also illustrates another possible feature of the present invention. Gripper 70 may or may not be provided with a slot 78 for allowing the gripper 70 to be placed on or removed from the catheter shaft 12. This slot 78 may be sized so that the gripper tends to “snap” into position around the catheter shaft 12.

The gripper is preferably made of a material with a high coefficient of friction. Possible examples such materials are rubbers, polyurethanes, polyetherblockamide. One example of the present gripper invention was made of injection molded liquid silicone rubber, available from Dow Coming with a trade name of Silastic 9280-60.

It should be understood that an unlimited number of configurations for the present invention could be realized. The foregoing discussion describes merely exemplary embodiments illustrating the principles of the present invention, the scope of which is recited in the following claims. Those skilled in the art will readily recognize from the description, claims, and drawings that numerous changes and modification can be made without departing from the spirit and scope of the invention.

1. A gripper and catheter system for treating a patient comprising:
a catheter shaft having a proximal end and a distal end; the shaft defining a longitudinal axis;
a hub affixed to the catheter shaft near its proximal end; the hub providing a handle for manipulating the catheter shaft, the hub having a larger size than an outer radial dimension of the catheter shaft;
a tubular gripper surrounding a portion of the catheter shaft, the gripper defining inner and outer surfaces; the gripper outer surface having a plurality of outwardly protruding ridges extending transversely in a ring around the outer surface of the gripper;
wherein the gripper is movable from an initial position to a desired position, at least a portion of the gripper being flexible so that it can be temporarily squeezed to cause at least a portion of the gripper inner surface to contact a portion of an outer surface of the catheter shaft to that the gripper can transmit frictional forces to the catheter shaft;
when the squeezing pressure is released, the gripper tends to resiliently return to its original shape; such that the gripper may be moved to a second desired position on the catheter shaft.

2. The gripper and catheter system of claim 1, wherein the gripper is made of a polymer material.

3. The gripper and catheter system of claim 1, wherein the catheter further comprises a balloon affixed to the catheter shaft near its distal end.

4. The gripper and catheter system of claim 1, wherein the gripper in an initial position contacts a portion of the hub.

5. The gripper and catheter system of claim 1, wherein a feature affixed to the hub in an initial position has a releasable interference fit with the gripper, which tends to releasably hold the gripper in the initial position.

6. The gripper and catheter system of claim 1, wherein the catheter further comprises a balloon affixed to the catheter shaft, and the catheter shaft defines an inflation lumen connecting an inflation port defined by the hub to an interior of the balloon, and the catheter shaft defines a guidewire lumen connecting a distal guidewire port distal of the balloon and a proximal guidewire port positioned between the balloon and the hub, in a rapid-exchange configuration.

7. The gripper and catheter system of claim 1, wherein the catheter shaft has a lubricious coating.

8. The gripper and catheter system of claim 1, wherein the gripper is releasably locked in an initial position by a snap-fit with the hub.

9. The gripper and catheter system of claim 1, wherein the hub and gripper have complementary screw threads, such that the gripper can be releasably held in an initial position.

10. The gripper and catheter system of claim 1, further comprising a strain relief tube affixed to the hub and catheter shaft, the gripper in an initial position surrounding at least a portion of the strain relief tube.

11. The gripper and catheter system of claim 10, wherein the strain relief tube comprises a protruding ring, and the gripper has a corresponding inner indented ring.

12. The gripper and catheter system of claim 1, wherein the gripper is made of a material with a high coefficient of friction.

13. The gripper and catheter system of claim 1, wherein an initial gap is defined between an outer dimension of the catheter shaft and an inner dimension of the gripper.

14. The gripper and catheter system of claim 1, wherein an outer dimension of the catheter shaft is approximately 2 millimeters or less.

15. The gripper and catheter system of claim 1, wherein the gripper is made of a material selected from the group: rubber, polyurethane silicone rubber, and PEBA.

16. The gripper and catheter system of claim 1, wherein the gripper inner surface is textured to enhance frictional contact with the catheter shaft.

17. A gripper and catheter system for treating a patient comprising:
a catheter shaft having a proximal end and a distal end; the shaft defining a longitudinal axis;
a hub affixed to the catheter shaft near its proximal end; the hub providing a handle for manipulating the catheter shaft, the hub having a larger size than an outer radial dimension of the catheter shaft;
a tubular gripper surrounding a portion of the catheter shaft, the gripper defining inner and outer surfaces;
wherein the gripper is movable from an initial position to a desired position, at least a portion of the gripper being flexible so that it can be temporarily squeezed to cause at least a portion of the gripper inner surface to contact a portion of an outer surface of the catheter shaft to that the gripper can transmit frictional forces to the catheter shaft;
when the squeezing pressure is released, the gripper tends to resiliently return to its original shape; such that the gripper may be moved to a second desired position on the catheter shaft;
wherein the gripper is initially affixed to the hub and the physician must initially break an attachment to move the gripper to the first desired gripping position.

18. A gripper and catheter system for treating a patient comprising:
a catheter shaft having a proximal end and a distal end; the shaft defining a longitudinal axis;
a hub affixed to the catheter shaft near its proximal end; the hub providing a handle for manipulating the catheter shaft, the hub having a larger size than an outer radial dimension of the catheter shaft;
a tubular gripper surrounding a portion of the catheter shaft, the gripper defining inner and outer surfaces;
wherein the gripper is movable from an initial position to a desired position, at least a portion of the gripper being flexible so that it can be temporarily squeezed to cause at least a portion of the gripper inner surface to contact a portion of an outer surface of the catheter shaft to that the gripper can transmit frictional forces to the catheter shaft;
when the squeezing pressure is released, the gripper tends to resiliently return to its original shape; such that the gripper may be moved to a second desired position on the catheter shaft;
wherein the gripper is made of more than one layer of different materials.

19. A gripper and catheter system for treating a patient comprising:
a catheter shaft having a proximal end and a distal end; the shaft defining a longitudinal axis;
a hub affixed to the catheter shaft near its proximal end; the hub providing a handle for manipulating the catheter shaft, the hub having a larger size than an outer radial dimension of the catheter shaft;
a tubular gripper surrounding a portion of the catheter shaft, the gripper defining inner and outer surfaces; the gripper having one or more slits formed in the gripper, so that it can be positioned around or removed from the catheter shaft;
wherein the gripper is movable from an initial position to a desired position, at least a portion of the gripper being flexible so that it can be temporarily squeezed to cause at least a portion of the gripper inner surface to contact a portion of an outer surface of the catheter shaft to that the gripper can transmit frictional forces to the catheter shaft;
when the squeezing pressure is released, the gripper tends to resiliently return to its original shape; such that the gripper may be moved to a second desired position on the catheter shaft.
